(19) United States
(12) Patent Application Publication
(10) Pub. No.: US 2019/0360055 A1
(43) Pub. Date: Nov. 28, 2019

(54) METHOD OF QUANTIFYING DNA

(71) Applicant: THE TRUSTEES OF INDIANA UNIVERSITY, Indianapolis, IN (US)

(72) Inventor: Tim Lautenschlaeger, Carmel, IN (US)

(21) Appl. No.: 16/420,307

(22) Filed: May 23, 2019

Related U.S. Application Data
(60) Provisional application No. 62/675,436, filed on May 23, 2018.

(51) Int. Cl. C12Q 1/6886; C12Q 1/6851; G16B 40/10; G16B 20/10; G16H 50/30

(52) U.S. Cl. CPC C12Q 1/6886; C12Q 1/6851; G16H 50/30; G16B 20/10; G16B 40/10

(57) ABSTRACT

The invention relates to cell survival assays. Specifically, the present invention relates to identification and quantification of cell-free DNA (cfDNA) for quantification of cell survival in a sample.

### CROSS-REFERENCE TO RELATED APPLICATIONS

This application claims the priority benefit under 35 U.S.C. § 119(e) of U.S. provisional patent application No. 62/675,436, filed May 23, 2018, the disclosure of which is incorporated by reference herein in its entirety.

### FIELD OF THE DISCLOSURE

The present disclosure relates to cell survival assays. In particular, the present disclosure relates to identification and quantification of cell-free DNA (cfDNA) in a sample for quantification of cell survival.

### BACKGROUND AND SUMMARY

Circulating tumor DNA (ctDNA) and cell-free DNA (cfDNA) analysis is a recent concept, which has been shown to aid in the detection of cancer and evaluation of somatic mutations, and is useful during cancer surveillance to potentially detect minimal residual disease with a clinically significant lead time between ctDNA detection and clinical relapse. CfDNA concentration in plasma increases in proportion to tumor volume and/or metabolic activity and growth, tumor necrosis, lymphovascular invasion and cellular proliferation indices.

CfDNA is fragmented in plasma and is thought to be derived from apoptotic and/or necrotic cells. The fragments are approximately 180 base-pairs in length following apoptosis or much larger following cell necrosis, and can be accurately quantified using digital droplet PCR (ddPCR). DdPCR is an exquisitely sensitive method to both quantify ctDNA/cfDNA and detect the presence of somatic mutations with an accuracy of about 1 in 10⁴ copies depending on the platform and number of wells and droplets. Radiation-induced cell death is known to occur predominantly by two pathways, apoptosis and mitotic catastrophe leading to cellular necrosis. As cancer cells grow, a small percentage of cells consistently die (conceptualized as cell loss factor) and release their intracytoplasmic and nuclear contents into plasma, in vivo, or into cell culture media, in vitro. Further, cfDNA release increases in breast cancer cell lines during G1 phase, in preparation for cell division, and a proportion of cfDNA is secreted actively via exosomes.

Radiotherapeutics, such as radiosensitizers, are useful in clinical practice, and there is a need for a reliable high-throughput method for measuring cell survival to expedite scientific discoveries from the bench to bedside. A novel cfDNA-based assay is described herein that estimates cell survival with high accuracy in multiple cell types. In addition, the assays described herein can be used in coculture models to accurately estimate the proportion of surviving colonies from different cell lines.

As described herein, cell-free DNA quantification may be used to measure cell survival, and is useful for high-throughput clonogenic assays and radiosensitizer screening platforms. The described methods show a strong correlation between CfDNA quantification from cell culture media and clonogenic survival at all radiation doses and in all cell lines tested. Cell survival curves derived from cfDNA are virtually indistinguishable from matched traditional clonogenic survival data (p 0.05; no significant difference exists between clonogenic curves). CfDNA quantification also accurately estimates colony count in a two cell-line coculture model as herein described.

As further described herein, the quantification of mutations in cell culture medium correlates with the number of growing cells in a sample. Thus, quantification of cell line specific mutations in cell culture medium is an alternative to/can replace traditional cell survival quantification methods. Pharmaceutical companies and academic laboratories may perform a variety of cell survival assays to screen drugs for efficacy. Previously described methods are expensive and labor intensive. The clonogenic assay has long been considered the standard to measure clonogenic survival in vitro after X-ray irradiation; however, it is limited by its labor-intensive nature. The novel assays described herein can be used for high throughput survival testing because multiple cell lines can be grown in the same culture vessel and assay read outs can be obtained earlier than through known standard assays.

In one embodiment, a method for quantification of cell survival in a sample is described, said method comprising: (a) extracting cell-free DNA (cfDNA) from a sample containing cells; (b) detecting the cfDNA in the sample; and (c) quantifying the number of living cells in the sample.

In one embodiment, the cells are contacted with a modulating agent. In various embodiments, the modulating agent is a radiotherapeutic agent or a chemotherapeutic agent. In one embodiment, the modulating agent is a radiosensitizer. In another embodiment, the cells are irradiated. In some embodiments, the cells are exposed to x-ray irradiation.

In one embodiment, the efficacy of a radiotherapeutic agent is determined by quantifying the number of living cells in the sample. In one embodiment, the efficacy of a chemotherapeutic agent is determined by quantifying the number of living cells in the sample.

In one embodiment, the cell-free DNA is circulating tumor DNA (ctDNA). In one embodiment, the cells are cancer cells. In some embodiment, the cancer cells are selected from the group consisting of cancer cells of the lung, bone, pancreas, liver, gallbladder, skin, uterus, ovary, endometrium, rectum, urethra, prostate, kidney, bladder, stomach, colon, breast, esophagus, small intestine, thyroid gland, parathyroid gland, and adrenal gland. In one embodiment, the cells are non-small-cell lung carcinoma cells. In some embodiments, the cells are selected from the group consisting of stem cells, pluripotent stem cells, progenitor cells, differentiated cells, beta cells, and fibroblasts.

In one embodiment, the cells are derived from a patient sample. In one embodiment, the cells are derived from an in vitro cell culture. In some embodiments, the cells are selected from the group consisting of adherent cultures, suspension cultures, single dissociated cells, and aggregated cells.

In one embodiment, the cfDNA in the sample comprises multiple extractions performed over multiple hours or days.

In one embodiment, detecting the cfDNA in the sample comprises quantifying the copy number of a gene in the cfDNA sample. In one embodiment, detecting the cfDNA in the sample comprises detecting a mutation in the cfDNA sample. In some embodiments, the gene copy number is quantified per ml of sample.

The various embodiments described in the numbered clauses below are applicable to any of the embodiments described in this “SUMMARY” section and the sections of the patent application titled “DETAILED DESCRIPTION OF ILLUSTRATIVE EMBODIMENTS” or “EXAMPLES” or in the “CLAIMS” appended to this application:

1. A method for quantification of cell survival in a sample, said method comprising: (a) extracting cell-free DNA (cfDNA) from a sample containing cells; (b) detecting the cfDNA in the sample; and (c) quantifying the number of living cells in the sample.

2. The method of clause 1, wherein the cells are contacted with a modulating agent.

3. The method of clause 2, wherein the modulating agent is a radiotherapeutic agent or a chemotherapeutic agent.

4. The method of clause 1, wherein the modulating agent is a radiosensitizer.

5. The method of clause 1, wherein the cells are irradiated.

6. The method of clause 5, wherein the cells are exposed to x-ray irradiation.

7. The method of clause 1, wherein the efficacy of a radiotherapeutic agent is determined by quantifying the number of living cells in the sample.

8. The method of clause 1, wherein the efficacy of a chemotherapeutic agent is determined by quantifying the number of living cells in the sample.

9. The method of clause 1, wherein the DNA is circulating tumor DNA (ctDNA).

10. The method of clause 1, wherein the cells are cancer cells.

11. The method of clause 10, wherein the cancer cells are selected from the group consisting of cancer cells of the lung, bone, pancreas, liver, gallbladder, skin, uterus, ovary, endometrium, rectum, urethra, prostate, kidney, bladder, stomach, colon, breast, esophagus, small intestine, thyroid gland, parathyroid gland, and adrenal gland.

12. The method of clause 11, wherein the cells are non-small-cell lung carcinoma cells.

13. The method of clause 1, wherein the cells are selected from the group consisting of stem cells, pluripotent stem cells, progenitor cells, differentiated cells, beta cells, and fibroblasts.

14. The method of clause 1, wherein the cells are derived from a patient sample.

15. The method of clause 1, wherein the cells are derived from an in vitro cell culture.

16. The method of clause 1, wherein the cells are selected from the group consisting of adherent cultures, suspension cultures, single dissociated cells, and aggregated cells.

17. The method of clause 1, wherein extracting the cfDNA from the sample comprises multiple extractions performed over multiple hours or days.

18. The method of clause 1, wherein detecting the cfDNA comprises quantifying the copy number of a gene in the cfDNA sample.

19. The method of clause 1, wherein detecting the cfDNA comprises detecting a mutation in the cfDNA sample.

20. The method of clause 18, wherein the gene copy number is quantified per ml of sample.

### BRIEF DESCRIPTION OF THE DRAWINGS

FIG. 1 shows that wild-type DNA degrades in media over time. A549 (KRAS mutant) cells were plated in two plating densities, fresh media was plated, and new media was collected at 0 (control), 14, 20, 38, and 44 hours, and the cfDNA was extracted and quantified. Due to A549 having a homozygous KRAS mutation, the KRAS WT DNA detected is intrinsic to the FBS in media. When using a wild-type DNA primer to quantify cell survival, background DNA reaches low levels after 38 hours, which is when it is appropriate to interpret ddPCR readouts in vitro, if interested in cell line derived cfDNA.

FIGS. 2A-C show that CfDNA in media directly correlates to plating density in H322 (FIG. 2A), H1299 (FIG. 2B), and A549 (FIG. 2C) cell lines. The X-axis represents each radiation dose the cells received (0, 4, or 8 Gy) as well as the plating density (i.e. number of cells plated/T25 plate). Cells were radiated and plated on T25 plates in duplicate, and the media was collected at 13 days post-XRT (H322) or 9 days (H1299/A549). CfDNA in culture media was extracted and quantified. Error bars shown represent the standard deviation between duplicate plates (n=2), and the bolded value represents the average copy number.

FIGS. 3A-C show that no significant difference exists between standard clonogenic curves to cfDNA-derived cell survival curves. The cfDNA derived cell survival curves' surviving fractions were calculated by generation a linear equation (colony count versus cfDNA Copy #) from cfDNA readouts at the highest and lowest plating densities in each cell line (FIG. 3A=H322, FIG. 3B=H1299, FIG. 3C=A549). FIG. 3A, comparison of cell survival curves in H322 cell line. FIG. 3B, comparison of cell survival curves in H1299 cell line. FIG. 3C, comparison of cell survival curves in A549 cell line. No significant differences exist between “cfDNA Derived SF” and “predicted SF” cell survival curves in all three cell lines (p>0.05).

FIGS. 4A-C show that CfDNA analysis strongly correlates with the relative colony count in a H1299:A549 coculture model. Each cell line was radiated to either 0 Gy (FIG. 4A), 4 Gy (FIG. 4B), or 8 Gy (FIG. 4C), and plated in coculture in varying proportions (0:100, 25:75, 50:50, 75:25, and 100:0(%)). CfDNA was isolated and quantified in culture media 9 days post-XRT by using KRAS WT (H1299) and KRAS Mutant (A549) primers. The Y-axis represents the KRAS copy number in cell culture media, and the X-axis represents the number of cells of each cell line plated. Due to differential release of cfDNA between cell lines in response to radiation and varying plating efficiencies between cell lines, the copy number from the A549-only plate was normalized to the H1299-only plate's copy number. All R2 values are >0.9 for each cell line and dose, and notably each line crosses at approximately 50:50 plating proportions, which agrees with our hypothesis. A=A549, H=H1299.

FIG. 5 shows that CfDNA quantification using ddPCR has low technical variability. H1299 cells were plated, and KRAS WT DNA was collected at time 0 (control) and after 22 h, allowing for cell turnover and cfDNA release into media. DdPCR technical replicates were then performed on two cfDNA samples in triplicate to determine intrinsic variability. CfDNA measurement at 0 h after X-ray irradiation had a standard deviation from the mean (SD)=4.97% while measurements at 22 h after X-ray irradiation revealed a SD=2.45%.

FIGS. 6A-C show that CfDNA in media is strongly correlated with clonogenic survival in three NSCLC cell lines. Cells were X-ray irradiated to 0 (control), 4 or 8 Gy and plated on T25 plates in duplicate. The media was collected at 9 days postirradiation (A549/H1299) or 13 days postirradiation (H322), and cfDNA in culture media was extracted and quantified. The number of DNA copies detected in media was compared to colony counts. The R2 values are depicted, and the dose is labeled in parentheses. FIG. 6A, H322 cell line; TP-53 mutant. FIG. 6B, H1299 cell line; KRAS WT. FIG. 6C, A549 cell line; KRAS mutant. FIG. 6A, in the H322 cell line, the R2 values for each XRT dose were 0.88 (0 Gy), 0.88 (4 Gy), and 0.82 (8 Gy) when combining all data points. FIG. 6B, in the H1299 cell line, the R2 values for each XRT dose were 0.95 (0 Gy), 0.99 (4 Gy), and 0.99 (8 Gy). FIG. 6C, in the A549 cell line, the R2 values for each XRT dose were 0.96 (0 Gy), 0.77 (4 Gy), and 0.99 (8 Gy). All cell lines and each experimental radiation dose show a strong correlation between cfDNA readouts and colony counts.

FIG. 7 shows that CfDNA in media closely estimates approximate colony counts for each cell line in coculture. H1299 and A549 were plated and irradiated to 0, 4 or 8 Gy, then replated in coculture in A549:H1299 ratios of 0:100, 25:75, 50:50, 75:25 or 100:0(%). “CfDNA derived colony counts at 50:50” were calculated by inputting cfDNA readout data into the linear equation (colony count versus cfDNA in media) from the H1299-only and A549-only plates. The “actual est. colony count” was calculated by extrapolating plating efficiencies of “H1299-only and A549-only plates”.

### DETAILED DESCRIPTION OF ILLUSTRATIVE EMBODIMENT

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

The present disclosure is generally related to methods of quantifying cell survival. In particular, the present disclosure relates to identification and quantification of cell-free DNA (cfDNA) for quantification of cell survival. In one embodiment, the methods as herein described are used for quantification of cell survival by determining the amount of cfDNA present in a sample. In another embodiment, gene copy number may be detected and/or quantified in a cfDNA sample. In another embodiment, a DNA mutation or multiple mutations may be detected and/or quantified in a cfDNA sample. Mutation detection can be performed using a variety of technologies (e.g., digital droplet PCR, next generation sequencing, or any method known in the art).

In one embodiment, a method for quantification of cell survival in a sample is described, said method comprising extracting cell-free DNA (cfDNA) from a sample containing cells, detecting the cfDNA in the sample, and quantifying the number of living cells in the sample. In one embodiment, a DNA quantification assay is described. In one embodiment, the DNA quantification assay is a cell-free assay. The assay may be an in vitro assay. In some embodiments, the cell survival estimation is performed using cfDNA. The assay may quantify cell survival following irradiation of the patient or cells.

Described herein is a novel method of measuring cell survival suitable for high-throughput survival analysis. As described, cell-line-specific DNA copies released in cell culture media consistently and accurately correlate with the number of colonies formed, even at low plating densities. This is consistent with the clinical observation that ctDNA concentration in plasma is directly related to tumor volume, metabolic activity and cellular proliferation index. As further described, a two-cell-line coculture model may be employed to estimate relative colony formation and radiosensitivity.

In one embodiment, the cells are contacted with a modulating agent. In various embodiments, the modulating agent is a radiotherapeutic agent or a chemotherapeutic agent. In one embodiment, the modulating agent is a radiosensitizer. In another embodiment, the cells are irradiated. In some embodiments, the cells are exposed to x-ray irradiation. The modulating agent may be any chemical compound, be it naturally-occurring or artificially-derived. In various embodiments, the modulating agent may include, without limitation, peptides, polypeptides, synthesized organic molecules, naturally occurring organic molecules, nucleic acid molecules, and radioisotopes.

The methods described herein are useful for identifying agents for use in chemotherapy or radiation therapy. The methods described herein may be used to determine an effective amount of an agent for chemotherapy or radiation therapy. Radiation therapy is a type of treatment (e.g., cancer treatment) that uses beams of intense energy to kill cancer cells. Radiation therapy most often uses X-rays, but protons or other types of energy can be used.

As used herein, an “effective amount” of a compound is the minimum amount of a compound that is necessary to minimally achieve, and more preferably, optimally achieve, the desired effect.

In one embodiment, the methods described herein may be used to screen the efficacy or toxicity of various chemotherapeutics or radiation therapies or to determine an effective amount of treatment, including, for example, external-beam radiation therapy (e.g., three-dimensional conformal radiation therapy, intensity-modulated radiation therapy, stereotactic radiosurgery (including Gamma Knife radiosurgery), stereotactic body radiation therapy, intraoperative radiation therapy, total body irradiation, electron therapy, proton therapy, and neutron therapy), internal radiation therapy (e.g., brachytherapy and systemic radiation therapy), or any means known in the art. For example, internal radiation therapy may be delivered via an implant, by mouth, or through intravenous delivery. Radiation therapy damages cells by destroying the genetic material that controls how cells grow and divide. While both healthy and cancerous cells are damaged by radiation therapy, the goal of radiation therapy is to destroy as few normal, healthy cells as possible. Normal cells can often repair much of the damage caused by radiation. Radiation therapy may be used to treat a patient, for example, as the primary treatment for cancer, before surgery to shrink a cancerous tumor (neoadjuvant therapy), after surgery to stop the growth of any remaining cancer cells (adjuvant therapy), in combination with other treatments, such as chemotherapy, to destroy cancer cells, and/or in advanced cancer to alleviate symptoms caused by the cancer.

The response of a cancer to radiation is described by its radiosensitivity. Highly radiosensitive cancer cells are rapidly killed by modest doses of radiation (e.g., leukemia, most lymphomas, and germ cell tumors). Many cancers are only moderately radiosensitive, and require a significantly higher dose of radiation (60-70 Gy) to achieve a radical cure (e.g., most epithelial cancers). Some types of cancer are notably radioresistant, that is, much higher doses are required to produce a radical cure than may be safe in clinical practice.

In one embodiment, a range of x-ray activation protocols may be employed to determine cytotoxic efficacy in relation to x-ray energy (kVp), total dose, and dose-rate. In one embodiment, kVp beam energies can range between 80 and 100 kV. In one embodiment, kVp beam energies can range from about 75 to about 125 kV, about 75 to about 150 kV, about 80 to about 160 kV, about 75 to about 200 kV, or about 80 to about 200 kV. In one embodiment, kVp beam energies can be about 50 kV, about 75 kV, about 80 kV, about 85 kV, about 90 kV, about 95 kV, about 100 kV, about 125 kV, about 145 kV, about 150 kV, about 155 kV, about 160 kV, about 165 kV, about 175 kV, or about 200 kV. kV beams may be obtained from various x-ray generating equipment, including orthovoltage units, standard diagnostic radiographic, fluoroscopic, and cone-beam computed tomography (CBCT) systems. In one embodiment, the x-ray dose may be relatively low (e.g., about 0.2Gy, 0.5Gy, or 1Gy). In one embodiment, the x-ray doses can range from about 0.2 to 2Gy. In various embodiments, the x-ray doses can range from about 0.1 to 2Gy, about 0.5 to 3Gy, about 0.5 to 4Gy, about 0.5 to 5Gy, about 0.5 to 6Gy, about 0.5 to 7Gy, about 0.5 to 8Gy, about 0.5 to 9Gy, about 1 to 9Gy, about 1 to 10Gy, about 2 to 5Gy, about 3 to 5Gy, about 3 to 8Gy, or about 3 to 10Gy. In one embodiment, the x-ray doses may be about 0.2Gy, 0.5Gy, 0.75Gy, 1.0Gy, 1.5Gy, 2.0Gy, 2.5Gy, 3.0Gy, 3.5Gy, 4.0Gy, 4.5Gy, 5.0Gy, 5.5Gy, 6.0Gy, 6.5Gy, 7.0Gy, 7.5Gy, 8.0Gy, 8.5Gy, 9.0Gy, 9.5Gy, 10Gy, 12Gy, 15Gy, 18Gy, or 20Gy.

For x-ray irradiation, the culture vessel may be positioned at a set distance (e.g., typically about 50 cm). In various embodiments, the culture vessel(s) may be set at a distance of about 10 cm, 20 cm, 30 cm, 40 cm, 50 cm, 55 cm, 60 cm, 65 cm, 70 cm, 75 cm, or 100 cm. Irradiations may be delivered in a “radiograph” mode; where multiple pulses of a set mA (e.g., typically 200 mA) and ms (e.g., typically 800 ms) and pulses can be delivered e.g., every 5-15 seconds. In one embodiment, the radiation can be delivered in a “pulsed fluoroscopy mode” (e.g., at 10 Hz) at the maximum mA setting. In one embodiment, kVp settings of 80 and 160 kVp (and ranges in between) with no added filtration in the beam (Half Value Layer=3.3 and 3.9 mm Al, respectively) are suitable. Higher kVps and lower kVps can be used.

As described herein, a test agent may interfere with modulation of apoptosis or cell death, cell proliferation, or with any biological response induced by the known compound. Generally, a test compound will exhibit at least 20% modulation, at least 25% modulation, or at least 30% to 50% modulation, 50 to 75% modulation, or 75% to 100% modulation. In some aspects, the compound may exhibit 50%, 60%, 70%, 75%, 80%, 85%, 90%, 95%, 98%, or 100% modulation when compared to untreated cells.

In various illustrative embodiments, candidate agents for modulating cell survival, or for the prevention or treatment of cell degenerative or cell proliferative disorders are identified from large libraries of both natural products or synthetic (or semi-synthetic) extracts or chemical libraries according to methods known in the art. Those skilled in the field of drug discovery and development will understand that the precise source of test extracts or compounds is not critical to the methods as herein described.

In one embodiment, the efficacy of a radiotherapeutic agent is determined by quantifying the number of living cells in the sample following irradiation. In one embodiment, the efficacy of a chemotherapeutic agent is determined by quantifying the number of living cells in the sample.

In one embodiment, the methods are used to identify a substance capable of modulating cell survival. In various embodiments, a substance or substances may be tested on a variety of cell types, either alone or in coculture, and cell survival determined. As used herein, “modulate” includes stimulation (e.g., increasing or upregulating a particular response or activity) and inhibition (e.g., decreasing or downregulating a particular response or activity). Modulation may be by direct or indirect means. In one embodiment, the methods are useful for identifying a substance capable of modulating cell survival and/or proliferation. In some embodiments, the methods described herein detect survival of a cell or cell population that displays abnormal properties of cell death, survival and/or proliferation regulation. In other embodiments, the methods detect survival of a cell or cell population that displays normal (as in typical) properties of cell death, cell survival and/or proliferation regulation. The assays of the present invention may be used, for example, to identify anti-tumor, angiogenisis-modulating and/or anti-inflammatory agents. The methods described herein are useful for carrying out high-throughput screening assays. In one embodiment, the substance used to modulate cell survival is radioactive (e.g., a radioisotope, x-rays, radiopharmaceuticals, etc.).

The ability of a substance to be cytotoxic can be determined by administering a candidate compound to cells and determining the % survival of said cells. The induction of cytotoxicity can be determined by various means. Further, there are several techniques known to a skilled person for determining if cell death is due to apoptosis, necrosis, or otherwise.

As described herein, cell survival refers to a reduction or decrease in cell death, or a promotion or increase in cell proliferation. Cell proliferation refers to excessive or aberrant cell growth. When the normal functions of cell survival go awry, the cause or the result can be cell degenerative or cell proliferative diseases, including cancer, viral infections, autoimmune disease/allergies, cardiovascular diseases, neurodegeneration, and related diseases.

In one embodiment, the cell-free DNA is circulating tumor DNA (ctDNA). In one embodiment, the cells are cancer cells. In various embodiments, the cancer cell may be derived from a carcinoma, a sarcoma, a lymphoma, a melanoma, a mesothelioma, a nasopharyngeal carcinoma, a leukemia, an adenocarcinoma, or a myeloma. In other embodiments, the cell may be from a lung cancer, bone cancer, pancreatic cancer, hepatobiliary cancer, cancer of the gallbladder, skin cancer, cancer of the head, cancer of the neck, cutaneous melanoma, intraocular melanoma, uterine cancer, ovarian cancer, endometrial cancer, rectal cancer, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, small cell lung cancer, cancer of the adrenal gland, sarcoma of soft tissue, cancer of the urethra, prostate cancer, penile cancer, testicular cancer, pancreatic endocrine cancer, carcinoid cancer, retinoblastomas, Hodgkin's lymphoma, non-Hodgkin's lymphomachronic leukemia, acute leukemia, a lymphocytic lymphoma, mesothelioma, cancer of the bladder, Burkitt's lymphoma, cancer of the ureter, cancer of the kidney, renal cell carcinoma, carcinoma of the renal pelvis, a neoplasm of the central nervous system (CNS), primary CNS lymphoma, a spinal axis tumor, a brain stem glioma, a pituitary adenoma, or an adenocarcinoma. In one embodiment, the cells are non-small-cell lung carcinoma cells.

In various embodiments, the cells are selected from the group consisting of stem cells, pluripotent stem cells, progenitor cells, differentiated cells, beta cells, and fibroblasts. Exemplary cells include stem cells, such as mouse embryonic stem cells, multiple neural stem cells, skin stem cells, mesenchymal stem cells, hematopoietic stem cells, stromal stem cells, and epithelial stem cells. Any type of progenitor cells can be used, including but not limited to, endoderm progenitor cells, mesoderm progenitor cells (e.g., muscle progenitor cells, bone progenitor cells, blood progenitor cells), and ectoderm progenitor cells (e.g., epidermal tissue progenitor cells and neural progenitor cells). In various embodiment, differentiated cells may be used. Differentiated cell populations may include, but are not limited to, fibroblasts, cardiac cells, neural cells, pancreatic beta cells, liver cells, epithelial cells, and intestinal cells. The cells described herein can be human cells or non-human cells. In some embodiments, the cells are human cells. In some embodiments, the cells are mouse, dog, cow, pig, rat or non-human primate cells.

In one embodiment, the cells are obtained from a population of cells and/or tissue. In one embodiment, the cells are obtained from a population of cells in culture. In one embodiment, the cells are obtained from a patient sample. Methods of obtaining cells and culturing cells derived from a patient sample (e.g., a body fluid or tissue) are known in the art. In one embodiment, a test agent is contacted to a population of cells prior to quantitating the cfDNA. In one embodiment, a modulating agent or any test agent is contacted to a population of cells prior to quantitating cell survival. Cells can be cultured according to any method known in the art. Cells can be cultured in suspension or as adherent cells as appropriate.

In one embodiment, the cells are derived from a patient sample. In one embodiment, cells can be isolated from tissues for in vitro or ex vivo culture. In one embodiment cells can be easily purified from patient fluid samples or solid tissues. In one embodiment, the cells may be derived from an explant culture.

As described herein, a “patient” can be a human or, in the case of veterinary applications, the patient can be a laboratory, an agricultural, a domestic, or a wild animal. In various aspects, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale. Exemplary patients include cancer patients, post-operative patients, transplant patients, patients undergoing chemotherapy, immunosuppressed patients, and the like. In one embodiment, the disease to be treated may be a transplant-associated disorder, an allergic disease, graft-versus-host disease, or an autoimmune disease.

In various illustrative embodiments, patient body fluids and tissues that can be used to obtain cells or DNA include, but are not limited to, whole blood; serum; plasma; urine; nasal secretions; bronchial washes, secretions, or aspirates; spinal fluid; bone marrow; sputum; pleural fluids; synovial fluids; pericardial fluids; peritoneal fluids; saliva; tears; gastric fluids; stool; reproductive tract secretions; and lymph fluid. These samples can be prepared for testing as described herein. In various embodiments, tissue samples can include tissue biopsies of hospital patients or out-patients and autopsy specimens. As used herein, the term “tissue” includes, but is not limited to, biopsies, autopsy specimens, cell extracts, tissue sections, aspirates, tissue swabs, and fine needle aspirates. In one aspect, cells can be obtained from a patient by means well-known in the art. For example, cells can be obtained by collecting peripheral blood from the patient or by collecting a tissue sample from the patient by means well-known in the art.

In one embodiment, the cells are derived from an in vitro cell culture. In one embodiment, cells may be obtained from cell lines or primary cells derived from a patient. Numerous cell lines are well established as representative of particular cell types and known in the art. For example, known cell lines available from American Type Culture Collection (ATCC) (Manassas, Va.) may be employed. In various embodiments, the cell lines may be human cell lines [e.g., DU145 (prostate cancer), H295R (adrenocortical cancer), HeLa (cervical cancer), KBM-7 (chronic myelogenous leukemia), LNCaP (prostate cancer), MCF-7 (breast cancer), MDA-MB-468 (breast cancer), PC3 (prostate cancer), SaOS-2 (bone cancer), SH-SYSY (neuroblastoma, cloned from a myeloma), T-47D (breast cancer), THP-1 (acute myeloid leukemia), U87 (glioblastoma)], primate cell lines [Vero (African green monkey kidney epithelial cell linen mouse cell lines [MC3T3 (embryonic calvarium)], rat cell lines [GH3 (pituitary tumor)], PC12 (pheochromocytoma)], plant cell lines [Tobacco BY-2 cells (model system of plant cell)], and the like.

As described herein, a cell culture refers to the removal of cells from an animal or plant and subsequent growth in a favorable environment. In one embodiment, the cells may be animal cells, plant cells, insect cells, bacterial cells, yeast cells, or fungal cells. The cells may be removed from a tissue directly and disaggregated by enzymatic or mechanical means before cultivation, or they may be derived from a cell line or cell strain that has already been established. Culture conditions vary widely for each cell type, but the environment in which the cells are cultured comprises one or more of a suitable culture vessel, a substrate or medium that supplies the essential nutrients (e.g., amino acids, carbohydrates, vitamins, minerals), growth factors, hormones, gases (e.g., O2, CO2), and a regulated physico-chemical environment (e.g., pH, osmotic pressure, temperature). Methods for growth of cells in culture are readily available and known to those skilled in the art.

In some embodiments, the cells are selected from the group consisting of adherent cultures, suspension cultures, single dissociated cells, and aggregated cells. In one embodiment, cells may be anchorage-dependent. Anchorage-dependent cells may be cultured while attached to a solid or semi-solid substrate (adherent or monolayer culture). In one embodiment, cells may grow in a suspension (i.e., grown floating in the culture medium).

The methods described herein can be used on cells obtained or grown in vitro, ex vivo, or in vivo. Suitable cells can include, but are not limited to, primary cells and established cell lines, embryonic cells, immune cells, stem cells, and differentiated cells including, but not limited to, cells derived from ectoderm, endoderm, and mesoderm, including fibroblasts, parenchymal cells, hematopoietic cells, and epithelial cells. Cells can include progenitor cells, unipotent cells, multipotent cells, and pluripotent cells; embryonic stem cells, inner mass cells, bone marrow cells, cells from umbilical cord blood. The cells can be ectoderm, mesoderm, or endoderm, or cells derived therefrom. The cells can be adult stem cells such as hematopoietic stem cells, mesenchymal stem cells, epithelial stem cells, and muscle satellite cells. In some embodiments the cells are induce pluripotent stem (iPS) cells. The cells can be stem cells, or progenitor cells, from various adult tissues including, but not limited to, neuronal/brain, cardiac muscle, skeletal muscle, gastrointestinal, skin, liver, kidney, adipose, etc.).

In some embodiments, the cells are cultured in contact with feeder cells. Exemplary feeder cells include, but are not limited to fibroblast cells. Methods of culturing cells on feeder cells are known in the art. In some embodiments, the cells are cultured in the absence of feeder cells. Cells, for example, can be attached directly to a solid culture surface (e.g., a culture plate), e.g., via a molecular tether. Exemplary molecular tethers include, but are not limited to, matrigel, an extracellular matrix (ECM), ECM analogs, laminin, fibronectin, or collagen. Those of skill in the art however will recognize that this is a non-limiting list and that other molecules can be used to attach cells to a solid surface.

In one embodiment, the cfDNA in the sample comprises multiple extractions performed over multiple hours or days. In one embodiment, the assay is validated after short growth periods, for example, such as after 7 days or after 9 days, to provide quicker experimental turnaround and thus further time and resource savings. In one aspect, the assay may be validated after a 7 day growth period, or after 1, 2, 3, 4, 5, 6, 8, 9, 10, 11, 12, 13, 14, or 15 days of growth, or longer if desired. In one aspect, the assay may be performed after 0, 14, 20, 38, or 44 hours of cell growth. In exemplary embodiments, the cells may be grown for 0, 1, 2, 3, 4, 5, 6, 7, 8, 12, 24, 30, 36, 40, 44, 48, 60, 72, 84, 96, 108, or 120 hours post modulation before the cell survival assay is performed. In various illustrative aspects, the cells may be plated for assay validation following 0, 2, 4, 6, 8, 10, 12, 14, 16, 18, 20, 22, 24, 26, 28, 30, 32, 34, 36, 38, 40, 44, 48, 60, 72, 84, 96, 108, or 120 hours post modulation. The modulating agent may be contacted with the cells when the cell culture is at, for example, about 25%, 30, 45%, 50%, 60%, 70%, 75%, 80%, 85%, 90%, 95%, or 100% confluence.

In one embodiment, the methods described herein may be used to estimate cell survival. For example, proliferating cells undergo a fixed amount of cell turnover and cfDNA is actively secreted in constant amounts via exosomes. This cell turnover and active cfDNA secretion consistently releases cfDNA into media in quantities proportional to the number of surviving cells. Cells that are not proliferating, such as cells which have become senescent due to radiation, will not contribute to the cfDNA readout.

In one embodiment, the methods described herein may be used to estimate cell survival in very low plating densities (for example, lower than that typically used in 96-well plates). In one aspect, the total number of cells and the concentration of the cells will vary depending on a number of factors including the type of cell used, single cell or coculture, type of cell growth (e.g. adhered growth or suspension), culture vessel type and size (e.g., single dish, T25 flask, 6-well, 12-well, 96-well plate, etc.). In one embodiment, the methods described herein are not dependent on plating density to enable accurate colony counting, which saves time and resources. In other embodiments, the plating densities may include but are not limited to from about 1.0×10² to about 1.0×10⁵ cells, about 1.0×10⁵ to about 1.0×10¹⁵ cells, or from about 1.0×10⁷ to about 1.0×10¹⁰ cells/ml. In various embodiments the plating density may be selected from the group consisting of about 0.5×10², 0.5×10³, about 1.0×10³, about 5.0×10³, about 0.5×10⁵, about 0.5×10⁶, about 1.0×10⁵, about 1.0×10⁶, about 5.0×10⁵, about 5.0×10⁶, about 0.5×10⁷, about 1.0×10⁷, about 5.0×10⁷, about 0.5×10¹⁰, about 1.0×10¹⁰, and about 5.0×10¹⁰ cells per culture vessel. In one embodiment, the cells are plated at 20, 40, 50, 60, 75, 80, 90, 100, 150, 200, 250, 300, 350, 400, 450, 500, 550, 750, 1000, 1200, 1500, 2000, 2500, 3000, 3500, 4000, 5000, 6000, 7000, 8000, 9000, 10,000, 12,000, 15,000, 16,000, 18,000, 20,000, 22,000, 25,000, 40,000, 50,000, or 75,000 cells/culture vessel.

As described herein, the cells may be primary cells isolated from a patient, or cells of an established cell line. The cells can be of a homogenous cell type, or can be a heterogeneous mixture of different cells types. For example, the cells can be from a heterogenous cell line possessing cells of different types, such as in a feeder cell culture, or a mixed culture in various states of differentiation. The cells can be a transformed cell line that can be maintained indefinitely in cell culture. The methods described herein are useful in the field of personalized therapy, for example, to determine the effective dose of a chemotherapeutic or radiotherapy prior to treatment of a patient.

In one embodiment, coculture assays may be employed. For example, a two-cell-line coculture may be used in the methods described herein. In cell lines that require coculture for survival (e.g., mouse or human embryonic stem cell lines that require feeder lines), cfDNA readouts may be used to more quickly and accurately estimate cell survival.

As described herein, an isolated cell has been substantially separated or purified away from other cells of an organism. The term dissociating cells refers to a process of isolating cells from other cells or from a surface (e.g., a culture plate surface). For example, cells can be dissociated from an animal or tissue by mechanical or enzymatic methods. Alternatively, cells that aggregate in vitro can be dissociated from each other. In yet another embodiment, adherent cells are dissociated from a culture plate or other surface. Dissociation thus can involve breaking cell interactions with extracellular matrix (ECM) and substrates (e.g., culture surfaces) or breaking the ECM between cells.

In one embodiment, detecting the cfDNA in the sample comprises quantifying the copy number of a gene in the cfDNA sample. In one embodiment, detecting the cfDNA in the sample comprises detecting a mutation in the cfDNA sample. In some embodiments, the gene copy number is quantified per ml of sample. The methods described herein can be used to detect or identify specific nucleic acid sequences in a DNA sample. Techniques for isolation of DNA are well-known in the art. Methods for isolating DNA are described in Sambrook et al., “Molecular Cloning: A Laboratory Manual”, 3rd Edition, Cold Spring Harbor Laboratory Press, (2001), incorporated herein by reference.

In the methods herein described, DNA may be detected and/or quantified using any DNA detection method known in the art. In one embodiment, the nucleic acid may be detected using conventional polymerase chain reaction (PCR) methods. In one embodiment, the nucleic acid may be detected using conventional polymerase chain reaction (PCR), quantitative PCR (qPCR), or digital PCR (dPCR). As described herein, PCR techniques may be used to amplify specific, target DNA fragments from low quantities of source DNA or RNA (for example, after a reverse transcription step to produce complementary DNA (cDNA). When performing conventional PCR, the final concentration of template is proportional to the starting copy number and the number of amplification cycles. In one embodiment, a given number of reactions is performed on a single sample and the result is an analysis of fragment sizes or, for quantitative real-time PCR (qPCR), the analysis is an estimate of the concentration of the target sequences in the reaction-based on the number of cycles required to reach a quantification cycle (Cq).

For qPCR methods, a fluorescent reporter dye is used as an indirect measure of the amount of nucleic acid present during each amplification cycle. The increase in fluorescent signal is directly proportional to the quantity of exponentially accumulating PCR product molecules (amplicons) produced during the repeating phases of the reaction. Reporter molecules may be categorized as; double-stranded DNA (dsDNA) binding dyes, dyes conjugated to primers, or additional dye-conjugated oligonucleotides, referred to as probes. The use of a dsDNA-binding dye, such as SYBR® Green I, represents the simplest form of detection chemistry. When free in solution or with only single-stranded DNA (ssDNA) present, SYBR Green I dye emits light at low signal intensity. As the PCR progresses and the quantity of dsDNA increases, more dye binds to the amplicons and hence, the signal intensity increases. Alternatively, a probe (or combination of two depending on the detection chemistry) can add a level of detection specificity beyond the dsDNA-binding dye, since it binds to a specific region of the template that is located between the primers. The most commonly used probe format is the Dual-Labeled Probe (DLP; also referred to as a Hydrolysis or TaqMan® Probe). The DLP is an oligonucleotide with a 5′ fluorescent label, e.g., 6-FAM™ and a 3′ quenching molecule, such as one of the dark quenchers e.g., BHQ®1 or OQ™ (see Quantitative PCR and Digital PCR Detection Methods). These probes are designed to hybridize to the template between the two primers and are used in conjunction with a DNA polymerase that has 5′ to 3′ exonuclease activity.

For digital PCR (dPCR), the sample can be diluted and separated into a large number of reaction chambers or partitions. In various embodiments, each partition contains either one copy of the target DNA or no copies of the target DNA. In some embodiments, the partition may contain one or more copies of the target DNA. In some embodiments, the partition may contain two or more copies of the target DNA. The number of reaction chambers or partitions varies between systems, from several thousand to millions. The PCR is then performed in each partition and the amplicon detected using a fluorescent label such that the collected data are a series of positive and negative results.

In one embodiment, the methods described herein use droplet digital PCR (ddPCR) technology. ddPCR is a method for performing digital PCR that is based on water-oil emulsion droplet technology. For example, a sample is fractionated into thousands of droplets (e.g., 10,000, 15,000, 20,000, 25,000, 30,000, 40,000, or 50,000 droplets, or more depending on the reaction to be performed), and PCR amplification of the template molecules occurs in each individual droplet. The droplets for use in ddPCR are typically nanoliter-sized droplets. ddPCR has a small sample requirement reducing cost and preserving samples.

As described herein, for methods employing ddPCR, the sample(s) may be partitioned into 20,000 nanoliter-sized droplets. This partitioning allows the measurement of thousands of independent amplification events within a single sample. ddPCR technology uses reagents and workflows similar to those used for most standard TaqMan probe-based assays. ddPCR allows the detection of rare DNA target copies, allows the determination of copy number variation, and allows the measurement of gene expression levels with high accuracy and sensitivity. Digital PCR is an end-point PCR method that is used for absolute quantification and for analysis of minority sequences against a background of similar majority sequences, e.g., quantification of somatic mutations. When using this technique, the sample is taken to limiting dilution and the number of positive and negative reactions is used to determine a precise measurement of target concentration. The digital PCR (dPCR) methods may be employed using emulsion beads (e.g., Bio-Rad QX100™ Droplet Digital™ PCR, ddPCR™ system and RainDance Technologies' RainDrop™ instrument). In an alternative format, the reactions may be run on integrated fluidic circuits (chips). These chips have integrated chambers and valves for partitioning samples and reaction reagents (e.g., BioMark™, Fluidigm).

Techniques for isolation of DNA are well-known in the art. In one embodiment, cells may be ruptured by using a detergent or a solvent, such as phenol-chloroform. In another embodiment, cells remain intact and cell-free DNA may be extracted. DNA may be separated from other components in the sample by physical methods including, but not limited to, centrifugation, pressure techniques, or by using a substance with affinity for DNA, such as, for example, silica beads. After sufficient washing, the isolated DNA may be suspended in either water or a buffer. In other embodiments, commercial kits are available, such as Quiagen™, Nuclisensm™, and Wizard™ (Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

In various embodiments described herein, primers and/or probes are used for amplification of the target DNA are oligonucleotides from about ten to about one hundred, more typically from about ten to about thirty or about twenty to about twenty-five base pairs long, but any suitable sequence length can be used. In illustrative embodiments, the primers and probes may be double-stranded or single-stranded, but the primers and probes are typically single-stranded. The primers and probes described herein are capable of specific hybridization, under appropriate hybridization conditions (e.g., appropriate buffer, ionic strength, temperature, formamide, or MgCl₂ concentrations), to a region of the target DNA. The primers and probes described herein may be designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference.

Also within the scope of the invention are nucleic acids complementary to the probes and primers described herein, and those that hybridize to the nucleic acids described herein or those that hybridize to their complements under highly stringent conditions. In accordance with the invention “highly stringent conditions” means hybridization at 65° C. in 5×SSPE and 50% formamide, and washing at 65° C. in 0.5×SSPE. Conditions for low stringency and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization occurs along the full-length of the nucleic acid.

In some embodiments, also included are nucleic acid molecules having about 60%, about 70%, about 75%, about 80%, about 85%, about 90%, about 95%, at least 96%, at least 97%, at least 98%, or at least 99% homology to the probes and primers described herein. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid sequence of interest. Algorithms for database searching are typically based on the BLAST software. In some embodiments, the percent identity can be determined along the full-length of the nucleic acid.

As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

Techniques for synthesizing the probes and primers described herein are well-known in the art and include chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, Calif. or Integrated DNA Technologies, Skokie, Ill.). Techniques for purifying or isolating the probes and primers described herein are well-known in the art. Such techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as restriction enzyme analysis or sequencing, to determine if the sequence of the primers and probes is correct.

In some embodiments, the assays are designed to screen large libraries, e.g., by automating the assay steps, and providing compounds from any convenient source, which are typically run in parallel (e.g., in microtiter formats or in microwell plates in robotic assays). In some embodiments, the screening assays can be conveniently carried out in single culture plates or flasks, or in multi-well plates (e.g., 6-well, 12-well, 24-well, 48-well, 96-well, or 384-well plates, etc.). In some embodiments, two or more candidate agents are tested in a single reaction mixture.

In one embodiment, the methods described herein may be used to rapidly screen the efficacy of radiotherapeutics, e.g., radiosensitizers, in multiple cell types or multiple cell lines, or using cells derived from a patient sample, including a patient body fluid or tissue.

In one embodiment, the cells used in the methods described herein contain a DNA mutation. For example, the cells may contain a naturally occurring DNA mutation. In one embodiment, the cells contain foreign DNA, for example, produced by transfection, transduction, infection, transformation, and the like. Methods for incorporating foreign DNA into cells are well-known in the art. Methods for creating a mutation(s) in DNA are also well-known in the art. The methods herein described may be used in combination or alongside alternative and traditional clonogenic assays.

Various embodiments of the present disclosure will be more fully understood from the examples shown below. The EXAMPLES do not exemplify the full scope of the disclosure, but provide various additional illustrative aspects of the invention described herein and are not intended to be limiting in any way.

### Example 1

### Cell Culture, Irradiation, Media Collection, and Clonogenic Assay

Lung NSCLC cell lines including H1299, A549, and H322 were obtained and grown in the recommended media per ATCC protocol with 10% HyClone™ Fetal Bovine Serum (FBS) and 1% Corning™ Cellgro™ penicillin-streptomycin solution. All cell lines that were used for experimentation were authenticated through Applied Biosystems in November, 2017 to verify genetic identity. Cells were grown in 37° C. and 5% CO₂ conditions, and were passaged at approximately 85% confluence for no more than 20 passages. All experimentation was performed on fresh cell lines with <5 passages. Prior to X-ray irradiation, cells were plated using standard plating and counting techniques using a Countess™ II Automated Cell Counter, with four cell counts averaged to ensure accuracy and consistency between samples. Cells were given 24 h for cell adhesion, irradiated to 0 (plates set at room temperature alongside the X-ray machine), 4, or 8 Gy using a 320 kVP Precision™ X-ray machine with a 2-mm aluminum filter; d=50 cm, 160 kV, dose rate=0.72 Gy/min. After 24 h postirradiation, the cells were trypsinized and plated onto T25 plates in varying cell densities with 6 ml of cell culture media using standard counting, serial dilution and plating techniques. Care was given to perform this portion of the assay quickly with minimal time outside of the incubator to prevent unnecessary cell death and cfDNA release into cell culture media. Cells were placed in a dedicated shelf of the incubator, untouched, for 9 days (H1299/A549) or 13 days (H322) prior to media collection and cfDNA extraction. Culture media was carefully pipetted off the plate, centrifuged at 500 RPM×7.5 min, and the supernatant was collected and immediately frozen at −80° C., leaving a liberal amount of supernatant as to not disturb the DNA-laden cell pellet. After media collection, plates were fixed and stained using an ETOH/5% crystal violet solution, colonies >50 cells were manually counted, and the surviving fraction was calculated for each dose. Colony counting was performed independently from examination of cfDNA quantification results to prevent confirmation bias.

### Example 2

### Wild-type DNA Degradation Assay

To determine the rate in which background DNA degrades in FBS containing growth media in vitro, 500,000 and 1,000,000 A549 cells (KRAS mutant) were plated on T75 plates, and the copy number of KRAS WT DNA in media was measured at 0, 14, 20, 38 and 44 h after plating and media change.

### Example 3

### Coculture Experiment

H1299 and A549 human Non-Small Cell Lung Cancer cells were plated in varying percentages [0:100, 25:75, 50:50, 75:25, 100:0(%)] on T25 plates, media was collected at 9 days for DNA extraction and analysis, plates were fixed and stained and colonies counted at this time. Standard plating, counting, radiation, and clonogenic assay techniques were employed. Estimated colony counts for each cell line and plating ratio were calculated by extrapolating the control plate colonies' clonogenic survival, and this estimate was compared to the cfDNA predicted colony counts.

### Example 4

### DNA Extraction

Four milliliter cell culture media samples containing cfDNA were thawed at room temperature. CfDNA was extracted using the QIAamp™ Circulating Nucleic Acid kit (Qiagen) and eluted in 50 ul of buffer.

### Example 5

### Digital Droplet Quantification of cfDNA

Primers for wild-type (WT) and mutant KRAS G12S as well as TP53 R248L genes for the H1299, A549 and H322 cell lines, respectively, were obtained from BioRad (Assay ID nos: dHsaCP2500589, dHsaCP2500588, dHsaCP2500503, dHsaCP2500502 respectively). Eluted DNA from the three cell lines was prepared and amplified under standard cycling conditions for the C1000 Touch™ with a 96-well reaction module thermocycler (BioRad). Droplets were then generated and read using the QX200™ droplet generator and QX200™ droplet reader (BioRad). The copy number per ml of cell culture media was calculated from the droplet readout.

### Example 6

### Development of a CfDNA-based Clonogenic Assay

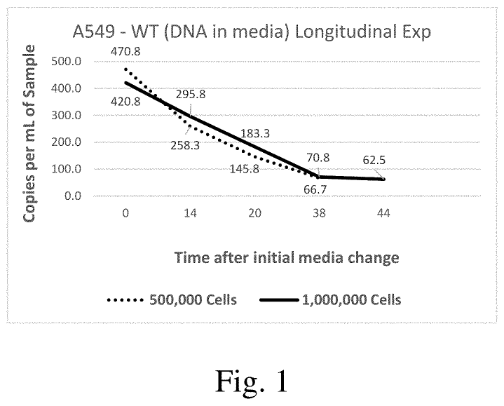

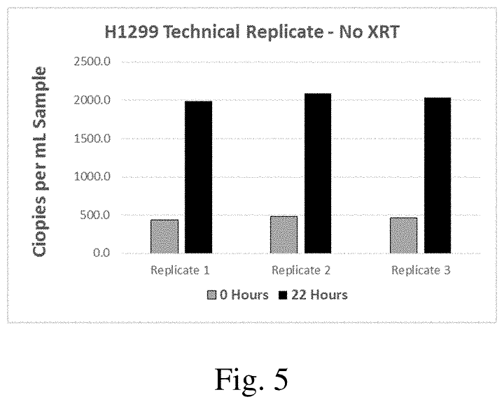

The time course of DNA degradation of the WT DNA in FBS containing growth media was determined to evaluate if WT DNA can be assayed in single cell line culture models. Background DNA reached low copy numbers of 70 KRAS WT copies/ml media after 38 h in both high and low plating densities (FIG. 1) and was effectively undetectable at 9 days after plating. To ensure reproducibility of ddPCR DNA quantification, cfDNA was quantified in triplicate from H1299 cell line media at 0 and 22 h after 8 Gy X-ray irradiation (n=3). DdPCR-based DNA quantification is highly reproducible with SD <5% (FIG. 5). Inter-plate variability, which includes cfDNA, DNA extraction, and ddPCR variability, is depicted below.

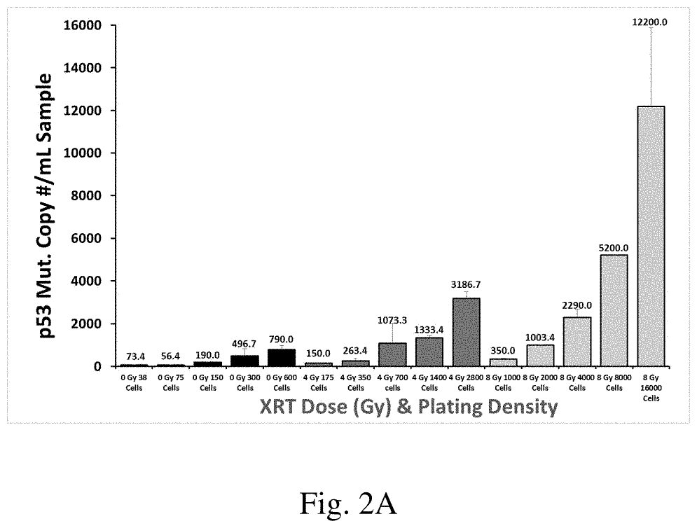

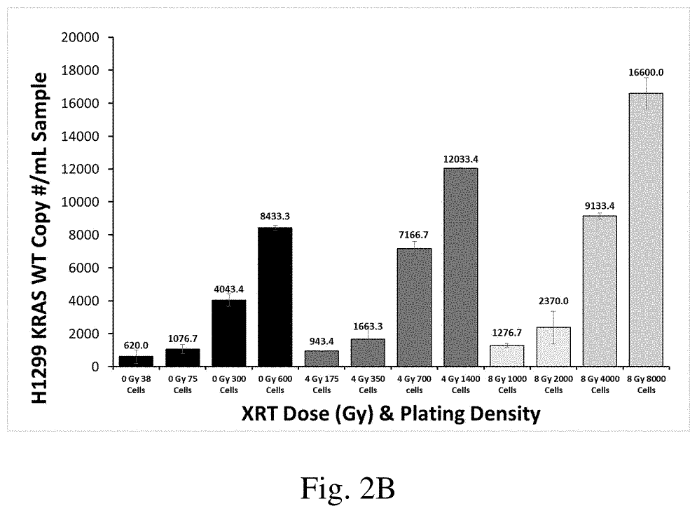

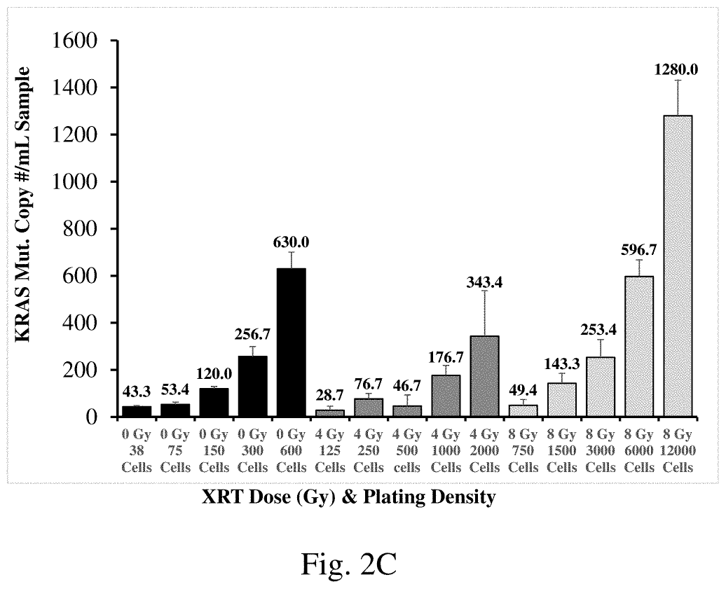

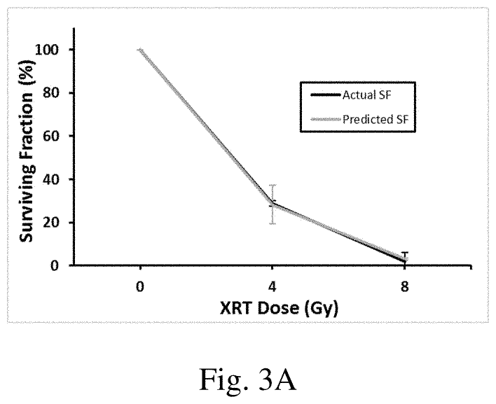

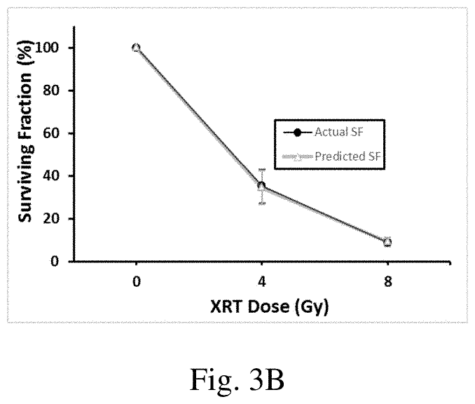

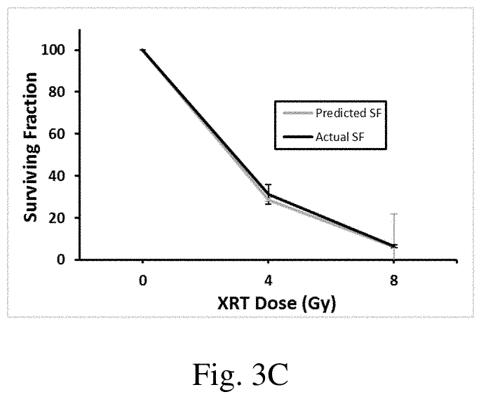

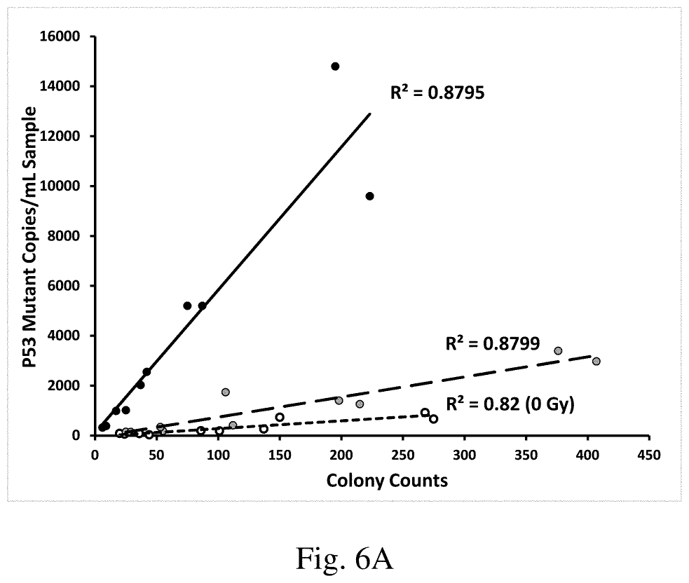

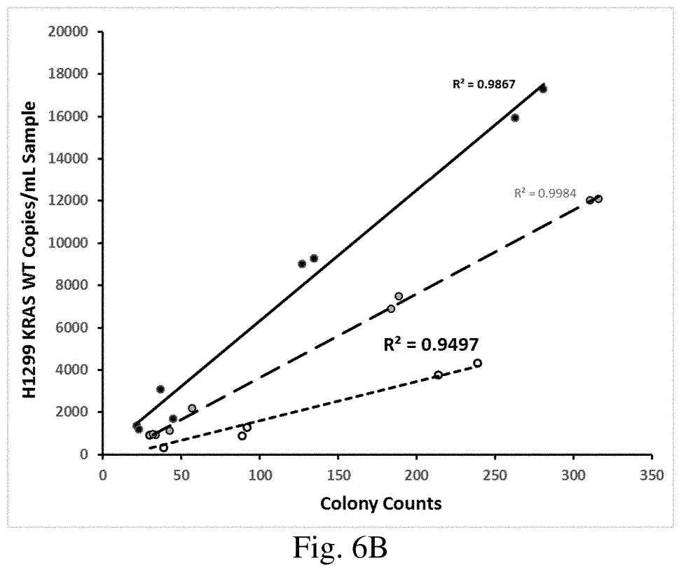

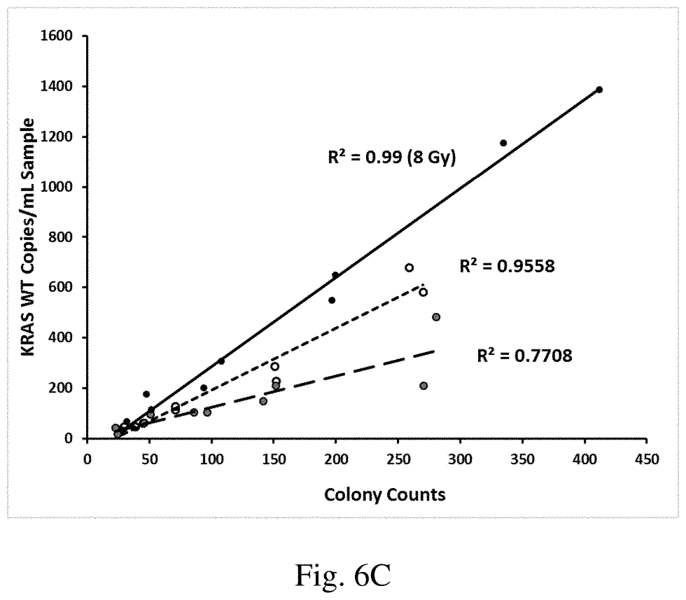

CfDNA copy number was also strongly correlated with colony counts in the H1299 cell line (KRAS WT) (FIG. 2B), with R2 values of 0.95 (0 Gy), 0.99 (4 Gy) and 0.99 (8 Gy) with all plating densities combined; n=2 (FIG. 6B). No significant difference between the “predicted clonogenic curve” and actual clonogenic curve was redemonstrated in this cell line; P>0.05 (FIG. 3B).

In the A549 cell line (KRAS mutant), a redemonstration of a strong correlation between cfDNA copy number and colony counts was exhibited (FIG. 2C), although the 4 Gy X-ray irradiated group had a weaker direct correlation. R2 values of 0.96 (0 Gy), 0.77 (4 Gy) and 0.99 (8 Gy) with all plating densities combined were calculated; n=2 plates/plating density (FIG. 6C). The actual and predicted clonogenic curves were not significantly different; P>0.05 (FIG. 3C).

One additional goal was to determine the smallest number of cells that could be precisely plated and still produce cfDNA in sufficient quantities to accurately be detected by ddPCR. Notably, the minimum number of cells plated in which cfDNA was accurately measured at 0, 4 and 8 Gy, respectively, was 38, 175 and 1,000 H322 and H1299 cells, and 38, 125, and 750 A549 cells (FIG. 2A-C).

### Example 7

### CfDNA Quantification Accurately Estimates Cell Line Proportions in Coculture

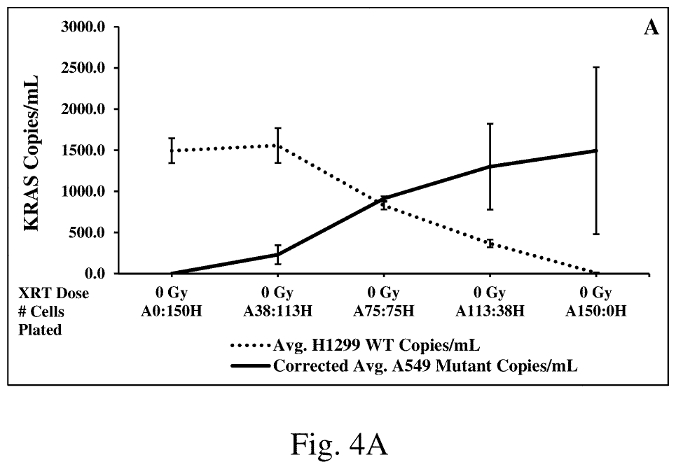

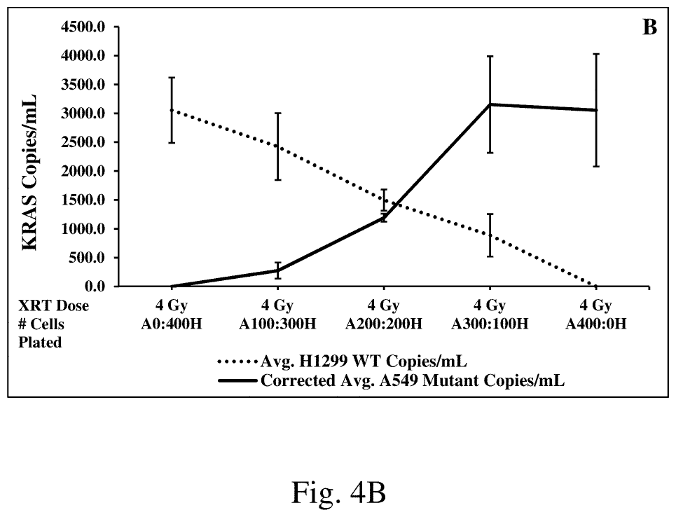

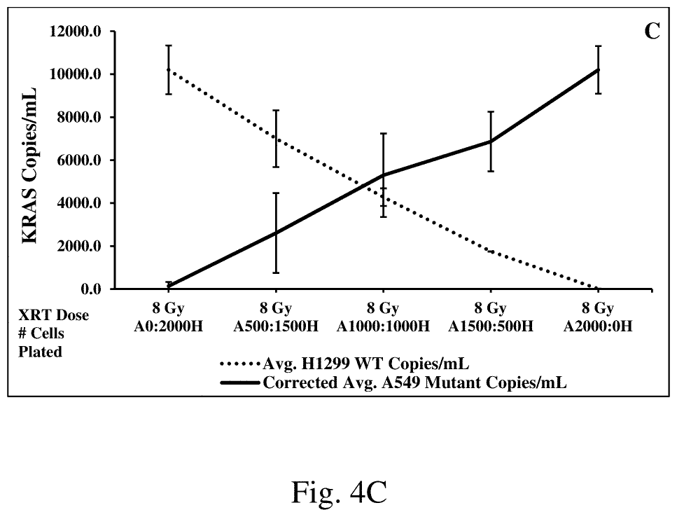

H1299 and A549 cocultures were produced in 0:100, 25:75, 50:50, 75:25, and 100:0 proportions, and cfDNA was isolated, and cell line-specific cfDNA was quantified in each plate following standard clonogenic assay protocol with 0, 4 or 8 Gy X-ray irradiation. Due to the intrinsic variation in cfDNA release between cell lines, the A549 cfDNA copy numbers were standardized to the H1299 control for direct comparison. In all groups, there was a strong direct correlation between the relative cell line plating density and the cfDNA quantified in coculture media (FIG. 4). The R2 values for each trendline comparing colony count versus cfDNA release in media were as follows: 0 Gy H1299=0.93; 0 Gy A549=0.96; 4 Gy H1299=0.99; 4 Gy A549=0.90; 8 Gy H1299=0.99; and 8 Gy A549=0.99.

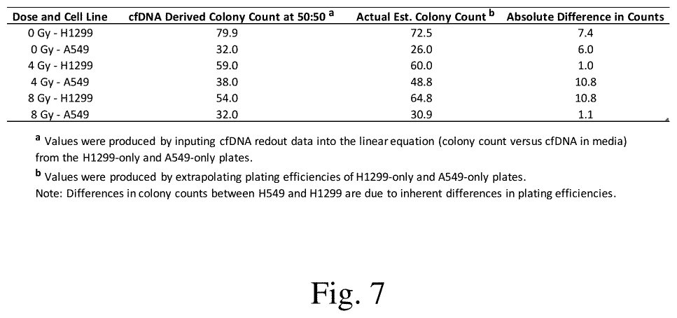

Furthermore, an estimated colony count was calculated by inputting [cfDNA] into the linear equation derived from the positive and negative controls (0:100 and 100:0 plates). The estimated cfDNA-derived colony counts closely estimated the actual estimated coculture colony counts (FIG. 7). These results indicate that cfDNA increases linearly with colony population size in a NSCLC coculture model, and cfDNA can be isolated and differentially quantified from multiple cell lines.

What is claimed is:

1. A method for quantification of cell survival in a sample, said method comprising:
extracting cell-free DNA (cfDNA) from a sample containing cells;
detecting the cfDNA in the sample; and
quantifying the number of living cells in the sample.

2. The method of claim 1, wherein the cells are contacted with a modulating agent.

3. The method of claim 2, wherein the modulating agent is a radiotherapeutic agent or a chemotherapeutic agent.

4. The method of claim 1, wherein the modulating agent is a radiosensitizer.

5. The method of claim 1, wherein the cells are irradiated.

6. The method of claim 5, wherein the cells are exposed to x-ray irradiation.

7. The method of claim 1, wherein the efficacy of a radiotherapeutic agent is determined by quantifying the number of living cells in the sample.

8. The method of claim 1, wherein the efficacy of a chemotherapeutic agent is determined by quantifying the number of living cells in the sample.

9. The method of claim 1, wherein the DNA is circulating tumor DNA (ctDNA).

10. The method of claim 1, wherein the cells are cancer cells.

11. The method of claim 10, wherein the cancer cells are selected from the group consisting of cancer cells of the lung, bone, pancreas, liver, gallbladder, skin, uterus, ovary, endometrium, rectum, urethra, prostate, kidney, bladder, stomach, colon, breast, esophagus, small intestine, thyroid gland, parathyroid gland, and adrenal gland

12. The method of claim 11, wherein the cells are non-small-cell lung carcinoma cells.

13. The method of claim 1, wherein the cells are selected from the group consisting of stem cells, pluripotent stem cells, progenitor cells, differentiated cells, beta cells, and fibroblasts.

14. The method of claim 1, wherein the cells are derived from a patient sample.

15. The method of claim 1, wherein the cells are derived from an in vitro cell culture.

16. The method of claim 1, wherein the cells are selected from the group consisting of adherent cultures, suspension cultures, single dissociated cells, and aggregated cells.

17. The method of claim 1, wherein extracting the cfDNA from the sample comprises multiple extractions performed over multiple hours or days.

18. The method of claim 1, wherein detecting the cfDNA comprises quantifying the copy number of a gene in the cfDNA sample.

19. The method of claim 1, wherein detecting the cfDNA comprises detecting a mutation in the cfDNA sample.

20. The method of claim 18, wherein the gene copy number is quantified per ml of sample.
